(19) United States
(12) Patent Application Publication
(10) Pub. No.: US 2015/0320391 A1
(43) Pub. Date: Nov. 12, 2015

(54) ULTRASONIC DIAGNOSTIC DEVICE AND MEDICAL IMAGE PROCESSING DEVICE

(71) Applicants: Kabushiki Kaisha Toshiba, Minato-ku (JP); Toshiba Medical Systems Corporation, Otawara-shi (JP)

(72) Inventors: Cong YAO, Otawara (JP); Shinichi HASHIMOTO, Otawara (JP)

(73) Assignees: Kabushiki Kaisha Toshiba, Minato-ku (JP); Toshiba Medical Systems Corporation, Otawara-shi (JP)

(21) Appl. No.: 14/802,496

(22) Filed: Jul. 17, 2015

Related U.S. Application Data
(63) Continuation of application No. PCT/JP2014/053657, filed on Feb. 17, 2014.

(30) Foreign Application Priority Data
Feb. 22, 2013 (JP) 2013-033654

(51) Int. Cl. A61B 8/00; A61B 17/34; A61B 8/06

(52) U.S. Cl. CPC A61B 8/4444; A61B 8/06; A61B 8/461; A61B 8/467; A61B 17/3403; A61B 2017/3413

(57) ABSTRACT

An ultrasonic diagnostic device according to an embodiment includes generation circuitry, detection circuitry, and display control circuitry. The generation circuitry generates an ultrasonic image based on reflected waves received by an ultrasonic probe. The detection circuitry detects an orientation of a puncture needle inserted into a subject. The display control circuitry displays the generated ultrasonic image while changing its orientation, corresponding to the detected orientation of the puncture needle.

### CROSS-REFERENCE TO RELATED APPLICATIONS

This application is a continuation of PCT international application Ser. No. PCT/JP2014/053657 filed on Feb. 17, 2014 which designates the United States, incorporated herein by reference, and which claims the benefit of priority from Japanese Patent Application No. 2013-033654, filed on Feb. 22, 2013, the entire contents of which are incorporated herein by reference.

### FIELD

Embodiments described herein relate generally to an ultrasonic diagnostic device and a medical image processing device.

### BACKGROUND

In the related art, widely used are ultrasonic diagnostic devices that transmit ultrasonic waves from the surface of a body to the interior of the body and generate an ultrasonic image based on reflected ultrasonic waves to easily observe the state of the interior of the body of a subject. For example, ultrasonic diagnostic devices can image the state of the interior of the body in substantially real time (including a case in which an image is generated with a slight time lag due to image generation processing time or a case in which static images are photographed one after another in a frame-by-frame manner) by pressing an ultrasonic probe that is relatively smaller than other diagnostic equipment against the surface of the body, so that the ultrasonic probe is used being brought into contact on the surface of the body in various orientations.

### BRIEF DESCRIPTION OF THE DRAWINGS

FIG. 1 is a diagram for explaining a configuration of an ultrasonic diagnostic device according to a first embodiment;

FIGS. 2A and 2B are a diagram for explaining a relation between an orientation of a display area and an orientation of an image;

FIGS. 3A and 3B are a diagram illustrating an example of an ultrasonic image displayed by an ultrasonic diagnostic device in the related art;

FIG. 4 is a diagram for explaining an angle component extracted by a display controller;

FIG. 5 is a diagram for explaining processing performed by a display controller according to the first embodiment;

FIG. 6 is a diagram for explaining the processing performed by the display controller according to the first embodiment;

FIG. 7 is a diagram for explaining the processing performed by the display controller according to the first embodiment;

FIG. 8 is a flowchart illustrating a processing procedure of the ultrasonic diagnostic device according to the first embodiment;

FIG. 9 is a diagram for explaining processing performed by a display controller according to a second embodiment;

FIG. 10 is a diagram for explaining processing performed by a display controller according to a third embodiment;

FIG. 11 is a diagram for explaining processing performed by a display controller according to a fourth embodiment;

FIG. 12 is a diagram for explaining the processing performed by the display controller according to the fourth embodiment;

FIG. 13 is a diagram for explaining the processing performed by the display controller according to the fourth embodiment;

FIGS. 14A and 14B are a diagram for explaining processing performed by an image generation unit and a display controller according to a fifth embodiment; and

FIG. 15 is a diagram for explaining a configuration of a medical information system according to a sixth embodiment.

### DETAILED DESCRIPTION

An ultrasonic diagnostic device according to an embodiment includes generation circuitry, detection circuitry, and display control circuitry. The generation circuitry generates an ultrasonic image based on reflected waves received by an ultrasonic probe. The detection circuitry detects an orientation of a puncture needle inserted into a subject. The display control circuitry displays the generated ultrasonic image while changing its orientation, corresponding to the detected orientation of the puncture needle.

The following describes an ultrasonic diagnostic device and a medical image processing device according to embodiments with reference to the drawings.

### First Embodiment

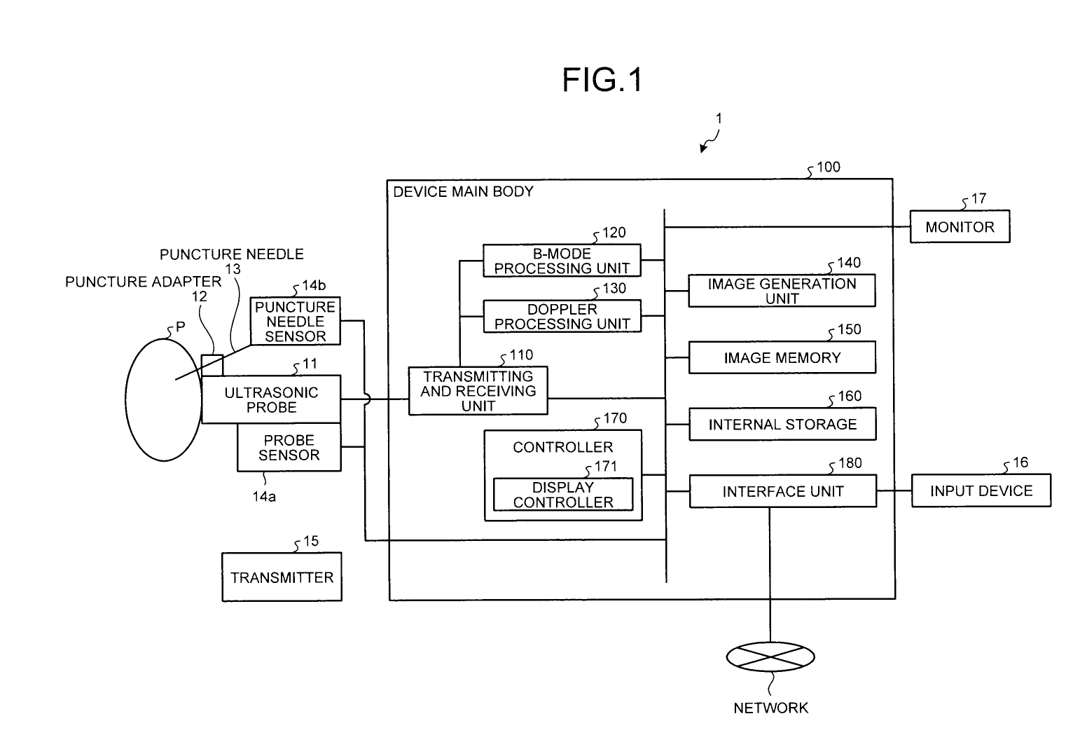

FIG. 1 is a diagram for explaining a configuration of an ultrasonic diagnostic device 1 according to a first embodiment. As illustrated in FIG. 1, the ultrasonic diagnostic device 1 according to the first embodiment includes an ultrasonic probe 11, a puncture needle 13, an input device 16, a monitor 17, and a device main body 100, and is connected to a network.

The ultrasonic probe 11 includes a plurality of piezoelectric transducer elements. These piezoelectric transducer elements generate ultrasonic waves based on a drive signal supplied from a transmitting and receiving unit 110 included in the device main body 100 (described later), and receive reflected waves from a subject P to be converted into electric signals. The ultrasonic probe 11 also includes a matching layer provided to the piezoelectric transducer element and a backing material that prevents the ultrasonic waves from propagating backward from the piezoelectric transducer element.

When the ultrasonic waves are transmitted from the ultrasonic probe 11 to the subject P, the transmitted ultrasonic waves are sequentially reflected by a discontinuous surface of acoustic impedance in body tissues of the subject P, and received as reflected wave signals by the piezoelectric transducer elements included in the ultrasonic probe 11. Amplitude of the received reflected wave signal depends on a difference in the acoustic impedance on the discontinuous surface by which the ultrasonic waves are reflected. When a transmitted ultrasonic pulse is reflected by a moving blood flow or a surface of a cardiac wall and the like, the reflected wave signals are subjected to frequency shift depending on a velocity component with respect to an ultrasonic wave transmitting direction of a mobile object due to the Doppler effect.

The embodiment can be applied to either of the following cases: a case in which the subject P is two-dimensionally scanned with the ultrasonic probe 11 serving as a one-dimensional ultrasonic probe in which the piezoelectric transducer elements are arranged in a line, or a case in which the subject P is three-dimensionally scanned with the ultrasonic probe 11 that mechanically shakes the piezoelectric transducer element in the one-dimensional ultrasonic probe (mechanical 4D probe) or the ultrasonic probe 11 serving as a two-dimensional ultrasonic probe in which the piezoelectric transducer element are two-dimensionally arranged in a grid shape (2D array probe).

A puncture adapter 12 is an attachment mounted on the ultrasonic probe 11 for performing puncturing at a certain position and orientation with respect to the ultrasonic probe 11. For example, the puncture adapter 12 has a groove (puncture guide rail) for attaching the puncture needle 13 at a certain position and orientation with respect to the ultrasonic probe 11. By way of example, the puncture guide rail is designed in advance so that the puncture needle 13 passes through a scanning plane scanned by the ultrasonic probe 11 from a position 5 cm away from a transmitting position of an ultrasonic beam at respective angles of 30 degrees, 45 degrees, and 60 degrees with respect to a transmitting direction of the ultrasonic beam.

In this case, the transmitting position and the transmitting direction of the ultrasonic beam indicate a transmitting position and a transmitting direction of a representative ultrasonic beam passing through the center of a scanning range, or an average transmitting position and an average transmitting direction of the ultrasonic beam in respective scanning lines. The puncture adapter 12 may be configured to automatically detect the puncture guide rail to which the puncture needle 13 is attached, and output the position and the angle of the puncture needle 13 with respect to the ultrasonic probe 11 to the device main body 100.

The puncture needle 13 is a medical tool that is attached to the puncture adapter 12 to perform puncturing for living tissue inspection, radiofrequency ablation treatment, and the like. For example, the puncture needle 13 passes through any of puncture guide rails on the puncture adapter 12 to be inserted into the subject P at a certain position and angle with respect to the ultrasonic probe 11. By way of example, a physician inserts the puncture needle 13 attached to the puncture adapter 12 into a region of interest (ROI) of the subject P while seeing an ultrasonic image displayed on the monitor 17.

A probe sensor 14a, a puncture needle sensor 14b, and a transmitter 15 are devices for acquiring an orientation of the ultrasonic probe 11 and an orientation of the puncture needle 13 in a real space. For example, the probe sensor 14a is a magnetic sensor installed in the ultrasonic probe 11 to detect the orientation of the ultrasonic probe 11 in the real space. For example, the puncture needle sensor 14b is a magnetic sensor installed in the puncture needle 13 to detect the orientation of the puncture needle 13 in the real space. For example, the transmitter 15 is a device that is provided at any position and forms a magnetic field toward the outside assuming that the transmitter itself is a center.

The probe sensor 14a and the puncture needle sensor 14b detect a three-dimensional magnetic field formed by the transmitter 15. The probe sensor 14a and the puncture needle sensor 14b then calculate coordinates and the angle of its own device in a space assuming that the transmitter 15 is an origin based on information about the detected magnetic field, and transmit the calculated coordinates and angle to a display controller 171 described later. In this case, the probe sensor 14a transmits the angle in the three-dimensional space at which its own device is positioned to the display controller 171 as the orientation of the ultrasonic probe 11. The puncture needle sensor 14b transmits the angle in the three-dimensional space at which its own device is positioned to the display controller 171 as the orientation of the puncture needle 13.

In the first embodiment, each of the probe sensor 14a and the puncture needle sensor 14b is not limited to the magnetic sensor. For example, the probe sensor 14a and the puncture needle sensor 14b may be gyroscopes (gyro sensors) that detect an angle or an angular speed of an object. That is, in the first embodiment, devices that can detect the orientation of the ultrasonic probe 11 and the orientation of the puncture needle 13 can be optionally applied to the probe sensor 14a and the puncture needle sensor 14b.

In the first embodiment, it is sufficient that the ultrasonic diagnostic device 1 includes at least one of the probe sensor 14a and the puncture needle sensor 14b. This is because the use of the puncture adapter 12 allows the puncture needle 13 to be attached to the ultrasonic probe 11 at a certain position and orientation, whereby the orientation of the ultrasonic probe 11 and that of the puncture needle 13 are interchangeable with each other.

The input device 16 includes a trackball, a switch, a button, a touch command screen, and the like, receives various instructions from an operator of the ultrasonic diagnostic device 1, and transfers the received various instructions to the device main body 100. For example, the input device 16 receives, from the operator, an instruction to insert the puncture needle 13 at a certain position and angle with respect to the ultrasonic probe 11 (instruction to select a puncture guide rail to be used in puncturing). The input device 16 stores, in an internal storage 160, the received position and angle of the puncture needle 13 with respect to the ultrasonic probe 11.

The monitor 17 displays a graphical user interface (GUI) through which the operator of the ultrasonic diagnostic device 1 inputs various instructions using the input device 16, or displays ultrasonic image data generated in the device main body 100 as an ultrasonic image.

The device main body 100 is a device that generates ultrasonic image data based on the reflected waves received by the ultrasonic probe 11. The device main body 100 illustrated in FIG. 1 can generate two-dimensional ultrasonic image data based on a two-dimensional reflected wave signal, and can generate three-dimensional ultrasonic image data based on a three-dimensional reflected wave signal.

As illustrated in FIG. 1, the device main body 100 includes the transmitting and receiving unit 110, a B-mode processing unit 120, a Doppler processing unit 130, an image generation unit 140, an image memory 150, the internal storage 160, a controller 170, and an interface unit 180.

The transmitting and receiving unit 110 controls transmission and reception of ultrasonic waves performed by the ultrasonic probe 11 based on an instruction from the controller 170 described later. The transmitting and receiving unit 110 includes a pulse generator, a transmission delay unit, a pulser, and the like, and supplies a drive signal to the ultrasonic probe 11. The pulse generator repeatedly generates rate pulses for forming transmission ultrasonic waves at a certain rate frequency. The transmission delay unit focuses the ultrasonic waves generated from the ultrasonic probe 11 into a beam, and gives, to each rate pulse generated by the pulse generator, a delay time for each piezoelectric transducer element required for determining transmission directivity. The pulser applies a drive signal (drive pulse) to the ultrasonic probe 11 at timing based on the rate pulse. The transmission delay unit changes the delay time to be given to each rate pulse to optionally adjust the transmitting direction of the ultrasonic waves transmitted from the surface of the piezoelectric transducer element.

The transmitting and receiving unit 110 also includes a preamplifier, an analog/digital (A/D) converter, a reception delay unit, an adder, and the like, and performs various pieces of processing on the reflected wave signal received by the ultrasonic probe 11 to generate reflected wave data. The preamplifier amplifies the reflected wave signal for each channel. The A/D converter executes A/D conversion on the amplified reflected wave signal. The reception delay unit gives a delay time required for determining reception directivity. The adder performs addition processing of the reflected wave signal processed by the reception delay unit, and generates the reflected wave data. Due to the addition processing performed by the adder, a reflection component from a direction corresponding to the reception directivity of the reflected wave signal is enhanced, and a comprehensive beam of transmission/reception of the ultrasonic waves is formed by the reception directivity and the transmission directivity.

To two-dimensionally scan the subject P, the transmitting and receiving unit 110 causes the ultrasonic probe 11 to transmit a two-dimensional ultrasonic beam. The transmitting and receiving unit 110 then generates two-dimensional reflected wave data from a two-dimensional reflected wave signal received by the ultrasonic probe 11. To three-dimensionally scan the subject P, the transmitting and receiving unit 110 causes the ultrasonic probe 11 to transmit a three-dimensional ultrasonic beam. The transmitting and receiving unit 110 then generates three-dimensional reflected wave data from a three-dimensional reflected wave signal received by the ultrasonic probe 11.

In this way, the transmitting and receiving unit 110 controls the transmission directivity and the reception directivity in transmitting/receiving the ultrasonic waves. The transmitting and receiving unit 110 has a function of instantly changing delay information, a transmission frequency, a transmission driving voltage, the number of aperture elements, and the like by being controlled by the controller 170 described later. In particular, to change the transmission driving voltage, used is a linear amplifier oscillator that can instantly switch a value, or a mechanism that electrically switches a plurality of power supply units. The transmitting and receiving unit 110 can transmit and receive different waveforms for each frame or each rate.

The B-mode processing unit 120 and the Doppler processing unit 130 are signal processing units that perform various pieces of signal processing on the reflected wave data generated from the reflected wave signal by the transmitting and receiving unit 110. The B-mode processing unit 120 receives the reflected wave data from the transmitting and receiving unit 110, and performs logarithmic amplification, envelope detection processing, and the like to generate data in which signal intensity is represented with brightness of luminance (B-mode data). The Doppler processing unit 130 performs frequency analysis of velocity information based on the reflected wave data received from the transmitting and receiving unit 110, and generates data (Doppler data) by extracting mobile object information such as velocity, distribution, power, and the like due to the Doppler effect at multiple points. In this case, examples of the mobile object include a blood flow, tissues such as a cardiac wall, and a contrast medium. The B-mode processing unit 120 and the Doppler processing unit 130 exemplified in FIG. 1 can process both of the two-dimensional reflected wave data and the three-dimensional reflected wave data.

The image generation unit 140 generates ultrasonic image data from the data generated by the B-mode processing unit 120 and the Doppler processing unit 130. That is, the image generation unit 140 generates, from the two-dimensional B-mode data generated by the B-mode processing unit 120, two-dimensional B-mode image data in which intensity of the reflected waves is represented with luminance. The image generation unit 140 also generates, from the two-dimensional Doppler data generated by the Doppler processing unit 130, two-dimensional Doppler image data representing mobile object information. The two-dimensional Doppler image data is velocity image data, distribution image data, power image data, or a combination thereof.

Typically, the image generation unit 140 converts a scanning line signal string for ultrasonic scanning into a scanning line signal string of a video format represented by a television and the like (scan-convert), and generates ultrasonic image data for display. Specifically, the image generation unit 140 performs coordinate transformation corresponding to a scanning mode of ultrasonic waves by the ultrasonic probe 11 to generate the ultrasonic image data for display. The image generation unit 140 also performs various pieces of image processing other than scan conversion, such as image processing using a plurality of scan-converted image frames to regenerate an average value image of luminance (smoothing processing), and image processing using a differential filter in an image (edge emphasis processing). The image generation unit 140 synthesizes the ultrasonic image data and accessory information (such as character information of various parameters, a scale, and a body mark).

That is, each of the B-mode data and the Doppler data is ultrasonic image data before scan-conversion processing, and the data generated by the image generation unit 140 is ultrasonic image data for display after scan-conversion processing. The B-mode data and the Doppler data are also called raw data. The image generation unit 140 generates “two-dimensional B-mode image data or two-dimensional Doppler image data” as two-dimensional ultrasonic image data for display from “two-dimensional B-mode data or two-dimensional Doppler data” as two-dimensional ultrasonic image data before scan-conversion processing.

The image generation unit 140 performs coordinate transformation on three-dimensional B-mode data generated by the B-mode processing unit 120 to generate three-dimensional B-mode image data. The image generation unit 140 also performs coordinate transformation on three-dimensional Doppler data generated by the Doppler processing unit 130 to generate three-dimensional Doppler image data. The image generation unit 140 generates “three-dimensional B-mode image data or three-dimensional Doppler image data” as “three-dimensional ultrasonic image data (volume data)”.

The image generation unit 140 performs rendering processing on the volume data to generate various pieces of two-dimensional image data for displaying the volume data on the monitor 17. Examples of rendering processing performed by the image generation unit 140 include processing of performing multi planer reconstruction (MPR) to generate MPR image data from the volume data. Examples of rendering processing performed by the image generation unit 140 also include volume rendering (VR) processing of generating two-dimensional image data reflecting three-dimensional information.

The image generation unit 140 generates image data for displaying a puncture guide line. For example, when the puncture adapter 12 is mounted on the ultrasonic probe 11, the image generation unit 140 obtains the position and the orientation of the puncture needle 13 with respect to the ultrasonic probe 11 from the internal storage 160 described later. Specifically, the image generation unit 140 obtains the fact that the puncture needle 13 is inserted from a position 5 cm away from the transmitting position of the ultrasonic beam at an angle of 45 degrees with respect to the transmitting direction of the ultrasonic beam on a scanning plane. The image generation unit 140 then generates image data for displaying the puncture guide line at position and orientation corresponding to the scanning range using the obtained position and orientation. The image generation unit 140 generates ultrasonic image data by superimposing the generated puncture guide line on the scanning range.

The image memory 150 stores image data such as a contrast image and a tissue image generated by the image generation unit 140. The image memory 150 also stores a processing result of the image generation unit 140. The image memory 150 stores an output signal immediately after passing through the transmitting and receiving unit 110, a luminance signal of the image, various pieces of raw data, image data acquired via a network, and the like as needed. A data format of the image data stored by the image memory 150 may be a format of data after video format conversion to be displayed on the monitor 17 by the controller 170 described later, or a format of data before coordinate transformation that is raw data generated by the B-mode processing unit 120 and the Doppler processing unit 130.

The internal storage 160 stores a control program for performing transmission/reception of ultrasonic waves, image processing, and display processing, diagnostic information (such as a patient ID and physician's findings), and various pieces of data such as a diagnostic protocol and various body marks. The internal storage 160 is also used to keep images stored by the image memory 150 as needed. The data stored by the internal storage 160 can be transferred to an external peripheral device via the interface unit 180 described later.

The internal storage 160 stores the position and the orientation of the puncture needle 13 inserted into the subject P from the puncture adapter 12 attached to the ultrasonic probe 11. For example, the internal storage 160 stores the fact that the puncture needle 13 is inserted from a position 5 cm away from the transmitting position of the ultrasonic beam at an angle of 45 degrees with respect to the transmitting direction of the ultrasonic beam on the scanning plane. The internal storage 160 also stores a display size of the monitor 17.

The controller 170 controls the entire processing in the ultrasonic diagnostic device 1. Specifically, the controller 170 controls processing in the transmitting and receiving unit 110, the B-mode processing unit 120, the Doppler processing unit 130, and the image generation unit 140, or controls the ultrasonic image data and the like stored by the image memory 150 to be displayed on the monitor 17 based on various instructions input by the operator via the input device 16, various control programs read from the internal storage 160, and various pieces of setting information.

The interface unit 180 is an interface that controls exchange of various pieces of information between the input device 16 or a network and the device main body 100.

The entire structure of the ultrasonic diagnostic device according to the first embodiment has been described above. With such a configuration, the ultrasonic diagnostic device 1 according to the first embodiment can display the ultrasonic image to be intuitively recognizable through the processing described in detail below.

In this case, an ultrasonic image generated by an ultrasonic diagnostic device in the related art is displayed so that the transmission direction of the ultrasonic beam transmitted from the ultrasonic probe corresponds to a downward direction of the monitor, for example. Due to this, a gravity direction in the image is not necessarily displayed correspond to the downward direction of a display area, which makes it difficult for a viewer to recognize the displayed image intuitively. The gravity direction indicates the direction in which the gravity of the earth acts.

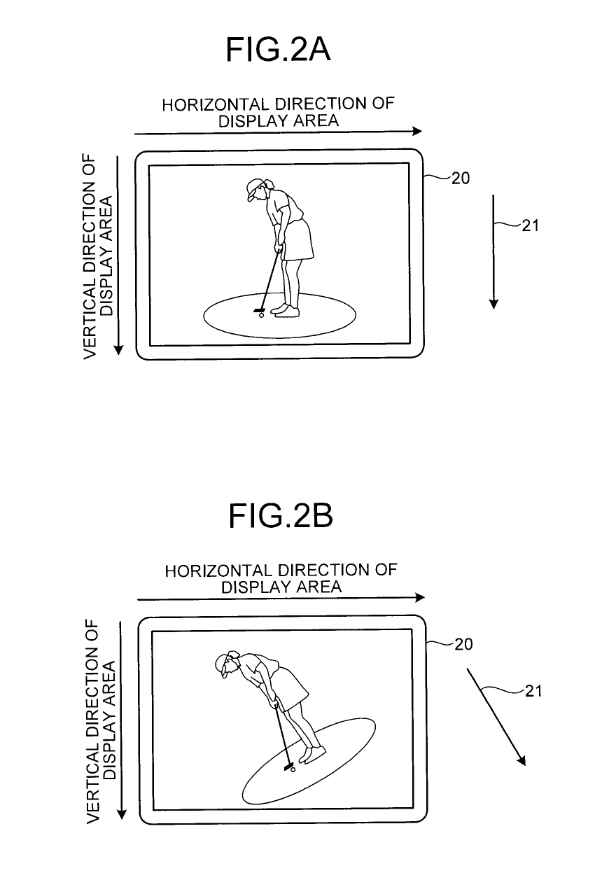

FIGS. 2A and 2B are diagrams for explaining a relation between the orientation of the display area and the orientation of the image. FIGS. 2A and 2B exemplify a case in which an image of a person playing golf is displayed in a display area of a display device 20. In FIGS. 2A and 2B, a right direction is assumed to be a positive direction along a horizontal direction of the display area, and a downward direction is assumed to be a positive direction along a vertical direction of the display area.

As illustrated in FIG. 2A, when a gravity direction 21 in the image is identical to the vertical direction of the display area, the display device 20 can display the image of a person playing golf so as to be intuitively recognizable to the viewer. In contrast, as illustrated in FIG. 2B, when the gravity direction 21 in the image is not identical to the vertical direction of the display area, the display device 20 cannot display the image of a person playing golf so as to be intuitively recognizable to the viewer, which gives a sense of incongruity to the viewer.

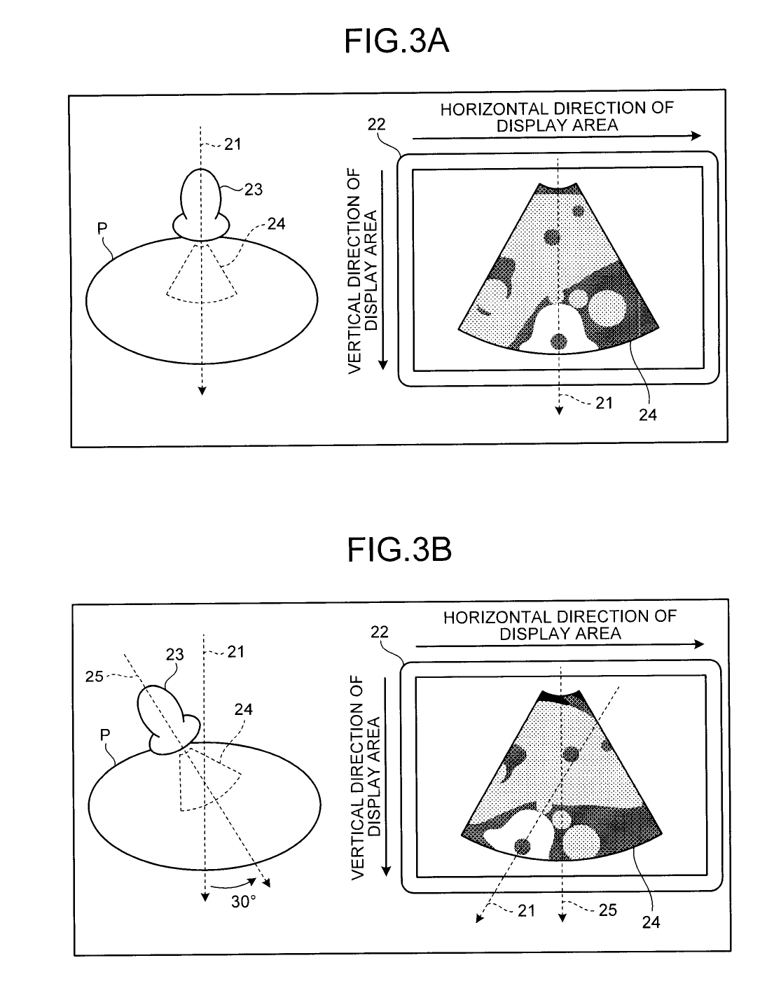

FIGS. 3A and 3B are diagrams illustrating an example of the ultrasonic image displayed by an ultrasonic diagnostic device in the related art. By way of example, FIGS. 3A and 3B exemplify a case in which an abdominal tomogram of the subject P is displayed on a monitor of an ultrasonic diagnostic device 22 in the related art. Specifically, the left figure in FIGS. 3A and 3B is an example of a positional relation between a transverse section of an abdominal region of the subject P lying on an inspection bed and an orientation of an ultrasonic probe 23 pressed against the abdominal region. The right figure in FIGS. 3A and 3B exemplifies a case in which the ultrasonic image obtained with the positional relation exemplified in the left figure is displayed in the display area of the monitor. In FIGS. 3A and 3B, the right direction is assumed to be the positive direction along the horizontal direction of the display area, and the downward direction is assumed to be the positive direction along the vertical direction of the display area.

As ultrasonic image is generated in a state where the ultrasonic probe 23 is pressed against the abdominal region of the subject P from directly above, the transmitting direction of the ultrasonic beam for scanning a scanning range 24 is identical to the gravity direction 21 in the real space. As illustrated in the right figure in FIG. 3A, the ultrasonic diagnostic device 22 in the related art displays the generated ultrasonic image with the transmitting direction of the ultrasonic beam for scanning the scanning range 24 being identical to the vertical direction of the display area. In this case, the gravity direction 21 in the ultrasonic image is identical to the vertical direction of the display area. That is, the orientation of the tomogram of the subject P displayed in the scanning range 24 is identical to the orientation of the subject P in the real space, so that the ultrasonic image in FIG. 3A is displayed to be intuitively recognizable to the viewer.

On the other hand, as illustrated in the left figure in FIG. 3B, when the ultrasonic image is generated in a state where the ultrasonic probe 23 is pressed against the abdominal region of the subject P at an angle of 30 degrees, a transmitting direction 25 of the ultrasonic beam for scanning the scanning range 24 is not identical to the gravity direction 21 in the real space, and is tilted by 30 degrees. In this case, as illustrated in the right figure in FIG. 3B, the ultrasonic diagnostic device 22 in the related art displays the generated ultrasonic image with the transmitting direction 25 of the ultrasonic beam for scanning the scanning range 24 being identical to the vertical direction of the display area. In this case, the gravity direction 21 in the ultrasonic image is not identical to the vertical direction of the display area, and is tilted by 30 degrees. That is, the orientation of the tomogram of the subject P displayed in the scanning range 24 is different from the orientation of the subject P in the real space, so that the ultrasonic image in FIG. 3B may give a sense of incongruity to the viewer.

In this way, the ultrasonic diagnostic device 22 in the related art changes the gravity direction of the ultrasonic image while being displayed depending on the orientation of the ultrasonic probe 23, which cannot always display the ultrasonic image so as to be intuitively recognizable. In addition, in the ultrasonic diagnostic device 22 in the related art, the transmitting direction 25 of the ultrasonic beam is changed by various angles every time the ultrasonic probe 23 is moved by the operator, so that the orientation of the tomogram of the subject P may be rotated by various angles.

Thus the ultrasonic diagnostic device 1 according to the first embodiment performs processing by the display controller 171 as described below so as to display the ultrasonic image that is intuitively recognizable.

The display controller 171 displays the generated ultrasonic image while changing its orientation, corresponding to the detected orientation of the ultrasonic probe 11 or the detected orientation of the puncture needle 13. For example, the display controller 171 receives the orientation of the ultrasonic probe 11 from the probe sensor 14a. Alternatively, the display controller 171 receives the orientation of the puncture needle 13 from the puncture needle sensor 14b. The display controller 171 then extracts an angle component corresponding to the horizontal direction of the ultrasonic probe 11 from the received orientation of the ultrasonic probe 11 or the received orientation of the puncture needle 13. Thus the display controller 171 displays the scanning range 24 of the ultrasonic image, generated by the image generation unit 140, being tilted by using the extracted angle component.

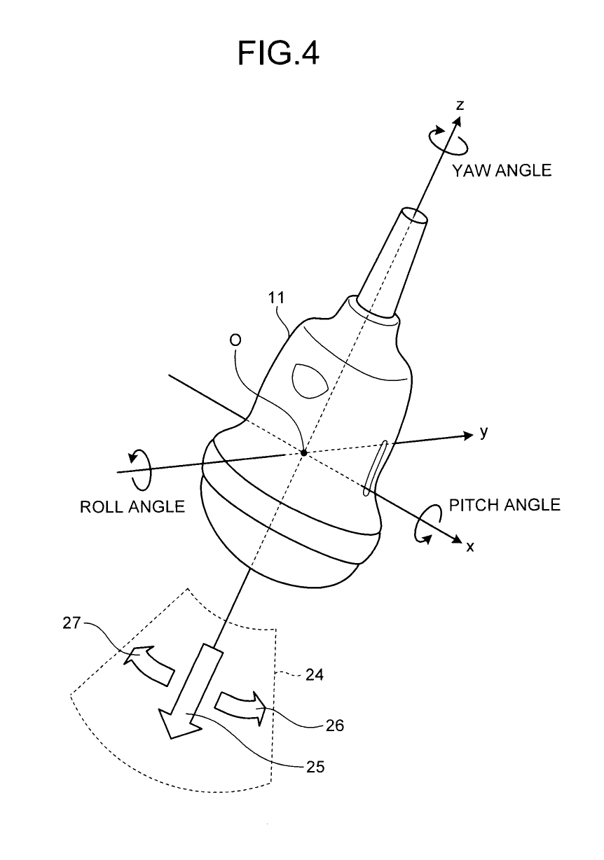

FIG. 4 is a diagram for explaining the angle component extracted by the display controller 171. FIG. 4 exemplifies the angle component of the ultrasonic probe 11 that can be detected by the probe sensor 14a or the puncture needle sensor 14b. In FIG. 4, the transmitting direction 25 of the ultrasonic beam transmitted from the ultrasonic probe 11 is assumed to be the z-axis. Any point passing through the z-axis inside the ultrasonic probe 11 is assumed to be an origin O. A direction passing through the origin O to be orthogonal to the scanning range 24 of the ultrasonic probe 11 is assumed to be the y-axis, and a direction orthogonal to the y-axis and the z-axis is assumed to be the x-axis. An angle around the x-axis is assumed to be a pitch angle, an angle around the y-axis is assumed to be a roll angle, and an angle around the z-axis is assumed to be a yaw angle. In such a coordinate system, the probe sensor 14a detects each of the pitch angle, the roll angle, and the yaw angle with respect to the gravity direction 21, and transmits the detected angle to the display controller 171 as the orientation of the ultrasonic probe 11.

As illustrated in FIG. 4, the display controller 171 receives each of the pitch angle, the roll angle, and the yaw angle with respect to the gravity direction 21 as the orientation of the ultrasonic probe 11. The display controller 171 extracts the roll angle from among received orientations of the ultrasonic probe 11 as the angle component corresponding to the horizontal direction of the ultrasonic probe 11. Hereinafter, an angle for rotating the transmitting direction 25 of the ultrasonic beam in a direction of an arrow 26 on the scanning plane is assumed to be a positive direction, and an angle for rotating the transmitting direction 25 in a direction of an arrow 27 is assumed to be a negative direction.

In the example of FIG. 4, described is a case in which the pitch angle, the roll angle, and the yaw angle detected by the probe sensor 14a are transmitted to the display controller 171. However, the embodiment is not limited thereto. For example, only the roll angle among the angles detected by the probe sensor 14a may be transmitted to the display controller 171.

In the example of FIG. 4, described is the orientation of the ultrasonic probe 11 detected by the probe sensor 14a. The orientation of the puncture needle 13 detected by the puncture needle sensor 14b is the same as the above orientation, and thus description thereof is not repeated here.

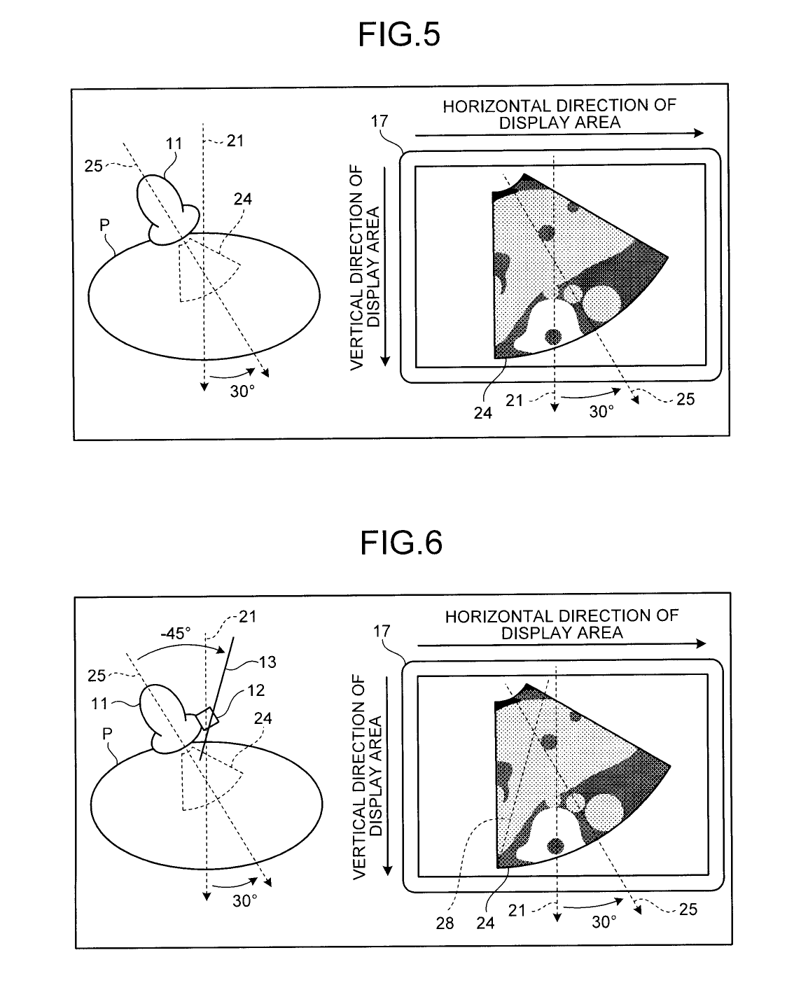

FIGS. 5 to 7 are diagrams for explaining processing performed by the display controller 171 according to the first embodiment. The left figure in FIGS. 5 to 7 is an example of a positional relation between the transverse section of the abdominal region of the subject P lying on the inspection bed and the orientation of the ultrasonic probe 11 pressed against the abdominal region. The right figure in FIGS. 5 to 7 exemplifies a case in which the ultrasonic image obtained with the positional relation exemplified in the left figure is displayed in the display area of the monitor. In FIGS. 5 to 7, the right direction is assumed to be the positive direction along the horizontal direction of the display area, and the downward direction is assumed to be the positive direction along the vertical direction of the display area.

With reference to FIG. 5, the following describes a case in which the display controller 171 displays the scanning range 24 of the ultrasonic image being tilted corresponding to the orientation of the ultrasonic probe 11. As illustrated in the left figure in FIG. 5, exemplified is a case in which the ultrasonic image is generated in a state where the ultrasonic probe 11 is pressed against the abdominal region of the subject P at the roll angle of “+30 degrees”. In this case, the display controller 171 extracts the roll angle of “+30 degrees” as the orientation of the ultrasonic probe 11.

As illustrated in the right figure in FIG. 5, the display controller 171 displays, on the monitor 17, the scanning range 24 of the ultrasonic image being tilted by the roll angle of “+30 degrees”. Specifically, the display controller 171 displays, on the monitor 17, a generated ultrasonic image, tilting the transmitting direction 25 of the ultrasonic beam for scanning the scanning range 24 by the roll angle of “+30 degrees” with respect to the vertical direction of the display area. More specifically, the display controller 171 transforms the coordinates of each pixel position included in the ultrasonic image data generated by the image generation unit 140, to display, on the monitor 17, the ultrasonic image being rotated by 30 degrees counterclockwise with respect to the display area of the monitor 17. As a result, the gravity direction 21 in the ultrasonic image is identical to the vertical direction of the display area. That is, the orientation of the tomogram of the subject P displayed in the scanning range 24 is identical to the orientation of the subject P in the real space (refer to FIG. 3A), and thus the ultrasonic image in FIG. 5 is displayed so as to be intuitively recognizable to the viewer.

With reference to FIG. 6, the following describes a case in which the display controller 171 displays the ultrasonic image on which a puncture guide line 28 is superimposed being tilted corresponding to the orientation of the ultrasonic probe 11. As illustrated in the left figure in FIG. 6, exemplified is a case in which the ultrasonic image is generated in a state where the ultrasonic probe 11 is pressed against the abdominal region of the subject P at the roll angle of “+30 degrees” and the puncture needle 13 is attached to the ultrasonic probe 11 at the roll angle of “−45 degrees”. In this case, the display controller 171 extracts the roll angle of “+30 degrees” as the orientation of the ultrasonic probe 11.

As illustrated in the right figure in FIG. 6, the display controller 171 displays, on the monitor 17, the scanning range 24 of the ultrasonic image on which the puncture guide line 28 is superimposed being tilted by the roll angle of “+30 degrees”. Specifically, the display controller 171 displays, on the monitor 17, the ultrasonic image on which the puncture guide line 28 is superimposed, tilting the transmitting direction 25 of the ultrasonic beam for scanning the scanning range 24 by the roll angle of “+30 degrees” with respect to the vertical direction of the display area. More specifically, the display controller 171 transforms the coordinates of each pixel position included in the ultrasonic image data on which the puncture guide line 28 is superimposed to display thereby, on the monitor 17, the ultrasonic image on which the puncture guide line 28 is superimposed being rotated by 30 degrees counterclockwise with respect to the display area of the monitor 17. As a result, the gravity direction 21 in the ultrasonic image is identical to the vertical direction of the display area. That is, the orientation of the tomogram of the subject P displayed in the scanning range 24 is identical to the orientation of the subject P in the real space (refer to FIG. 3A), and the orientation of the puncture guide line 28 is identical to an insertion angle of the puncture needle 13 in the real space, so that the ultrasonic image in FIG. 6 is displayed to be intuitively recognizable to the viewer.

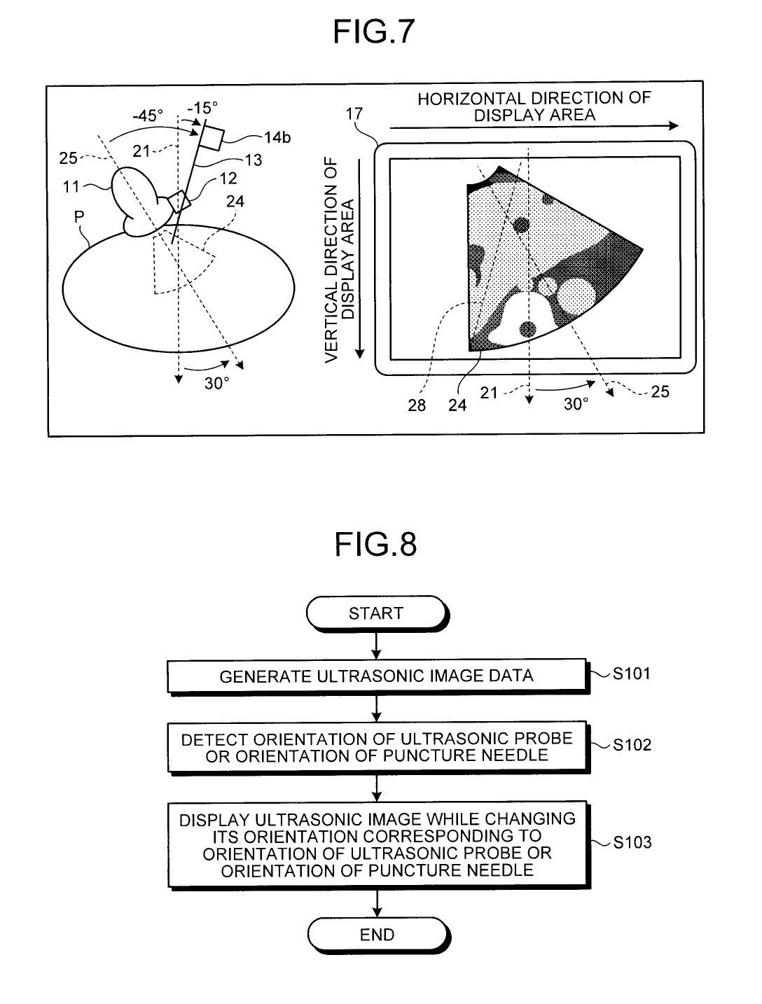

With reference to FIG. 7, the following describes a case in which the display controller 171 displays the scanning range 24 of the ultrasonic image being tilted corresponding to the orientation of the puncture needle 13. As illustrated in the left figure in FIG. 7, exemplified is a case in which the ultrasonic image is generated in a state where the ultrasonic probe 11 is pressed against the abdominal region of the subject P at the roll angle of “+30 degrees” and the puncture needle 13 is attached to the ultrasonic probe 11 at the roll angle of “−45 degrees”. That is, in this state, the puncture needle 13 is inserted into the abdominal region of the subject P at the roll angle of “−15 degrees” with respect to the gravity direction 21. In this case, the display controller 171 receives the orientation of the puncture needle 13 from the puncture needle sensor 14b. For example, the display controller 171 receives, from the puncture needle sensor 14b, the fact that the puncture needle 13 is inserted at the roll angle of “−15 degrees” with respect to the gravity direction 21 as the orientation of the puncture needle 13. The display controller 171 then extracts the roll angle of “−15 degrees” from the received orientation of the puncture needle 13. The display controller 171 receives, from the input device 16, the fact that the puncture needle 13 is inserted at the roll angle of “−45 degrees” with respect to the ultrasonic probe 11. At this point, the input device 16 has already received, from the operator, an instruction to insert the puncture needle 13 at the roll angle of “−45 degrees” with respect to the ultrasonic probe 11. The display controller 171 subtracts the roll angle of “−45 degrees” of the puncture needle 13 with respect to the ultrasonic probe 11 from the roll angle of “−15 degrees” of the puncture needle 13 with respect to the gravity direction 21 to calculate the roll angle of “+30 degrees” of the ultrasonic probe 11 with respect to the gravity direction.

As illustrated in the right figure in FIG. 7, the display controller 171 displays, on the monitor 17, the scanning range 24 of the ultrasonic image on which the puncture guide line 28 is superimposed being tilted by the roll angle of “+30 degrees”. Specifically, the display controller 171 displays, on the monitor 17, the ultrasonic image on which the puncture guide line 28 is superimposed, tilting the transmitting direction 25 of the ultrasonic beam for scanning the scanning range 24 by the roll angle of “+30 degrees” with respect to the vertical direction of the display area. More specifically, the display controller 171 transforms the coordinates of each pixel position included in the ultrasonic image data on which the puncture guide line 28 is superimposed to display, on the monitor 17, the ultrasonic image on which the puncture guide line 28 is superimposed being rotated by 30 degrees counterclockwise with respect to the display area of the monitor 17. As a result, the gravity direction 21 in the ultrasonic image is identical to the vertical direction of the display area. That is, the orientation of the tomogram of the subject P displayed in the scanning range 24 is identical to the orientation of the subject P in the real space (refer to FIG. 3A), and the orientation of the puncture guide line 28 is identical to the insertion angle of the puncture needle 13 in the real space, and thus the ultrasonic image in FIG. 7 is displayed so as to be intuitively recognizable to the viewer.

In this way, the display controller 171 extracts the angle component corresponding to the horizontal direction of the ultrasonic probe 11 from the orientation of the ultrasonic probe 11 or the orientation of the puncture needle 13, and uses the extracted angle component to display the scanning range 24 of the ultrasonic image being tilted.

FIG. 8 is a flowchart illustrating a processing procedure of the ultrasonic diagnostic device 1 according to the first embodiment. As illustrated in FIG. 8, in the ultrasonic diagnostic device 1 according to the first embodiment, the image generation unit 140 generates the ultrasonic image data (Step S101). The probe sensor 14a or the puncture needle sensor 14b detects the orientation of the ultrasonic probe 11 or the orientation of the puncture needle 13 (Step S102). The display controller 171 displays the ultrasonic image while changing its orientation, corresponding to the orientation of the ultrasonic probe 11 or the orientation of the puncture needle 13 (Step S103).

As described above, the ultrasonic diagnostic device 1 according to the first embodiment generates the ultrasonic image based on the reflected waves received by the ultrasonic probe. The ultrasonic diagnostic device 1 then detects the orientation of the ultrasonic probe 11 or the orientation of the puncture needle 13 in the real space. The ultrasonic diagnostic device 1 then displays the generated ultrasonic image while changing its orientation, corresponding to the orientation of the ultrasonic probe 11 or the orientation of the puncture needle 13. Due to this, the ultrasonic diagnostic device 1 can display the ultrasonic image to be intuitively recognizable.

For example, every time the ultrasonic probe 11 is moved by the operator, the ultrasonic diagnostic device 1 displays the ultrasonic image being tilted by the roll angle corresponding to the movement. Due to this, the ultrasonic diagnostic device 1 displays the ultrasonic image with the orientation of the tomogram of the subject P displayed in the scanning range 24 being identical to the orientation of the subject P in the real space regardless of the direction in which the ultrasonic probe 11 is oriented, and thus the ultrasonic image can be displayed so as to be intuitively recognizable to the viewer.

The ultrasonic diagnostic device 1 according to the first embodiment does not necessarily include the whole configuration illustrated in FIG. 1. Specifically, to perform processing of displaying the scanning range 24 of the ultrasonic image being tilted corresponding to the orientation of the ultrasonic probe 11 (processing of FIG. 5), the ultrasonic diagnostic device 1 does not necessarily include the puncture adapter 12, the puncture needle 13, and the puncture needle sensor 14b. To perform processing of displaying the ultrasonic image on which the puncture guide line 28 is superimposed being tilted (processing of FIG. 6), the ultrasonic diagnostic device 1 does not necessarily include the puncture needle sensor 14b. To perform processing of displaying the scanning range 24 of the ultrasonic image being tilted corresponding to the orientation of the puncture needle 13 (processing of FIG. 6), the ultrasonic diagnostic device 1 does not necessarily include the probe sensor 14a.

### Second Embodiment

In the first embodiment, described is a case in which puncturing is performed using the puncture adapter 12. However, the embodiment is not limited thereto. For example, the ultrasonic diagnostic device 1 can change the orientation of the ultrasonic image even when the puncturing is performed without using the puncture adapter 12, that is, when free-puncturing is performed. In the second embodiment, described is processing of changing the orientation of the ultrasonic image by the ultrasonic diagnostic device 1 to perform free-puncturing.

The configuration of the ultrasonic diagnostic device 1 according to a second embodiment is basically the same as the configuration of the ultrasonic diagnostic device 1 described with reference to FIG. 1. However, the configuration of the ultrasonic diagnostic device 1 according to the second embodiment is different from the configuration of the ultrasonic diagnostic device 1 described with reference to FIG. 1 in that the ultrasonic diagnostic device 1 according to the second embodiment includes both of the probe sensor 14a and the puncture needle sensor 14b, and part of the processing performed by the image generation unit 140 and the display controller 171 is different therebetween. Thus different points between the second embodiment and the first embodiment will be described, and the same points thereof will not be repeated.

The image generation unit 140 according to the second embodiment has the same function as that described in the first embodiment. The image generation unit 140 according to the second embodiment generates the ultrasonic image in which the puncture guide line 28 representing a puncture path of the puncture needle 13 is superimposed on the scanning range of the ultrasonic probe 11 using the position and the orientation of the ultrasonic probe 11 and the position and the orientation of the puncture needle 13.

For example, the image generation unit 140 obtains the position and the orientation of the ultrasonic probe 11 from the probe sensor 14a, and also obtains the position and the orientation of the puncture needle 13 from the puncture needle sensor 14b. The image generation unit 140 projects the position of the puncture needle 13 on the scanning plane to generate image data for displaying the puncture guide line 28. Subsequently, the image generation unit 140 calculates an intersection point of the scanning plane and the puncture guide line 28 in the scanning range 24. The image generation unit 140 then generates the ultrasonic image in which the puncture guide line 28 is superimposed on the scanning range 24 so that the puncture guide line 28 in front of the scanning plane (intersection point) of the scanning range 24 is indicated by a solid line, and the puncture guide line 28 at the back thereof is indicated by a dashed line. The intersection point is thus calculated because the puncture needle 13 does not necessarily present on the scanning plane in free-puncturing.

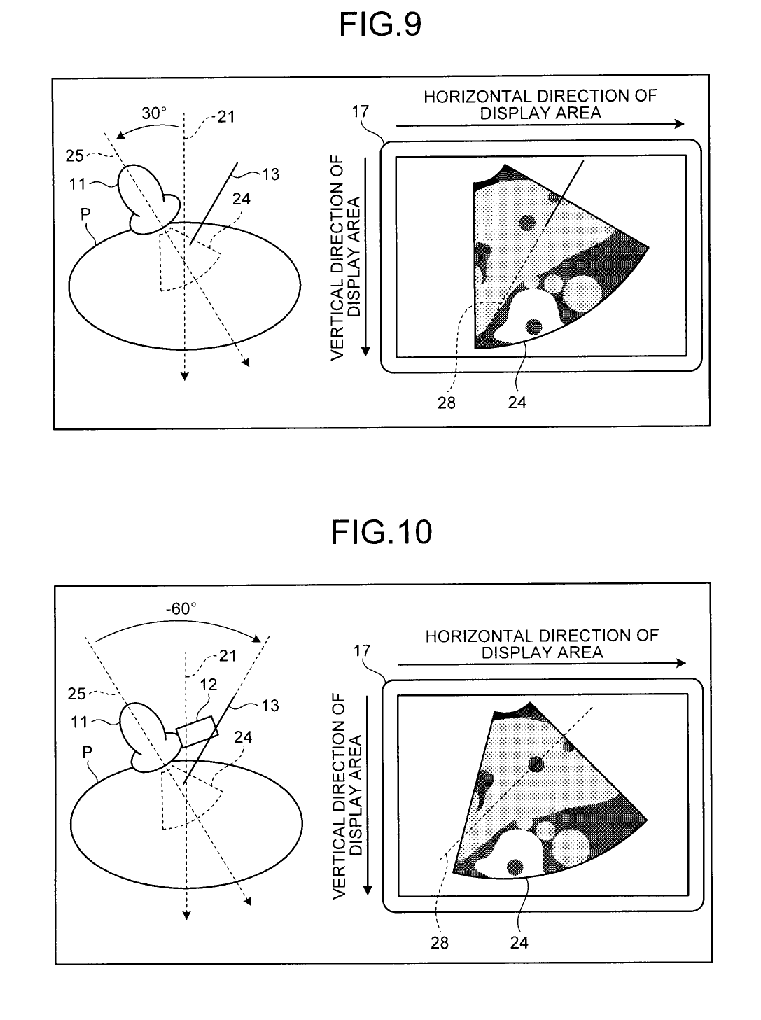

FIG. 9 is a diagram for explaining processing performed by the display controller 171 according to the second embodiment. The left figure in FIG. 9 is an example of a positional relation between the transverse section of the abdominal region of the subject P lying on the inspection bed and the orientation of the ultrasonic probe 11 pressed against the abdominal region. The right figure in FIG. 9 exemplifies a case in which the ultrasonic image obtained with the positional relation exemplified in the left figure is displayed in the display area of the monitor. In FIG. 9, the right direction is assumed to be the positive direction along the horizontal direction of the display area, and the downward direction is assumed to be the positive direction along the vertical direction of the display area.

As illustrated in the left figure in FIG. 9, exemplified is a case in which the ultrasonic image is generated in a state where the ultrasonic probe 11 is pressed against the abdominal region of the subject P at the roll angle of “+30 degrees”. In this case, the display controller 171 extracts the roll angle of “+30 degrees” as the orientation of the ultrasonic probe 11.

As illustrated in the right figure in FIG. 9, the display controller 171 displays, on the monitor 17, the scanning range 24 of the ultrasonic image on which the puncture guide line 28 is superimposed being tilted by the roll angle of “+30 degrees”. As a result, the gravity direction 21 in the ultrasonic image is identical to the vertical direction of the display area. That is, the orientation of the tomogram of the subject P displayed in the scanning range 24 is identical to the orientation of the subject P in the real space (refer to FIG. 3A), and the orientation of the puncture guide line 28 is identical to the insertion angle of the puncture needle 13 in the real space, so that the ultrasonic image in FIG. 9 is displayed to be intuitively recognizable to the viewer.

A case in which the display controller 171 displays the ultrasonic image being tilted using the orientation of the ultrasonic probe 11 has been described. However, the embodiment is not limited thereto. For example, the display controller 171 may display the ultrasonic image being tilted using the orientation of the puncture needle 13 and the positional relation between the ultrasonic probe 11 and the puncture needle 13. In this case, the positional relation between the ultrasonic probe 11 and the puncture needle 13 can be calculated from the position and the orientation of the ultrasonic probe 11 and the position and the orientation of the puncture needle 13.

As described above, the ultrasonic diagnostic device 1 according to the second embodiment detects the position and the orientation of the ultrasonic probe 11 with respect to the gravity direction using the probe sensor 14a, and detects the position and the orientation of the puncture needle 13 with respect to the gravity direction using the puncture needle sensor 14b. The ultrasonic diagnostic device 1 then generates the ultrasonic image in which the puncture guide line 28 representing the puncture path of the puncture needle 13 is superimposed on the scanning range 24 of the ultrasonic probe 11 using the position and the orientation of the ultrasonic probe 11 and the position and the orientation of the puncture needle 13. The ultrasonic diagnostic device 1 displays the ultrasonic image being tilted using the orientation of the ultrasonic probe 11 or the orientation of the puncture needle 13. Accordingly, the ultrasonic diagnostic device 1 can display the ultrasonic image so as to be intuitively recognizable even when free-puncturing is performed.

### Third Embodiment

In the above embodiment, described is a case in which the ultrasonic image is displayed in the orientation in the real space. However, the embodiment is not limited thereto. For example, the ultrasonic diagnostic device 1 can display the ultrasonic image being tilted so that the orientation of the puncture guide line is at a display angle intended by the operator. In a third embodiment, described is a case in which the ultrasonic diagnostic device 1 displays the ultrasonic image being tilted so that the orientation of the puncture guide line is at the display angle intended by the operator.

The configuration of the ultrasonic diagnostic device 1 according to the third embodiment is basically the same as the configuration of the ultrasonic diagnostic device 1 described with reference to FIG. 1. However, the configuration of the ultrasonic diagnostic device 1 according to the third embodiment is different from the configuration of the ultrasonic diagnostic device 1 described with reference to FIG. 1 in that the ultrasonic diagnostic device 1 according to the third embodiment does not need to include both of the probe sensor 14a and the puncture needle sensor 14b. Thus different points between the third embodiment and the first embodiment will be described, and the same points thereof will not be repeated.

The input device 16 according to the third embodiment receives, from the operator, an instruction on the display angle for displaying the puncture guide line 28 that indicates the puncture path of the puncture needle 13. For example, the input device 16 receives, from the operator, an instruction to display the puncture guide line 28 at the roll angle of “−45 degrees” with respect to the vertical direction of the display area. The input device 16 then transmits the display angle instructed by the operator to the display controller 171.

Similarly to the first embodiment, the input device 16 receives, from the operator, an instruction to insert the puncture needle 13 at a certain position and orientation with respect to the ultrasonic probe 11. For example, the input device 16 receives, from the operator, the instruction that the puncture needle 13 is attached to the ultrasonic probe 11 at the roll angle of “−60 degrees” from a position 5 cm away from the transmitting position of the ultrasonic beam. The input device 16 transmits, to the display controller 171, the position and the orientation of the puncture needle 13 with respect to the ultrasonic probe 11 instructed by the operator.

The display controller 171 according to the third embodiment displays the ultrasonic image being tilted corresponding to the instructed display angle.

FIG. 10 is a diagram for explaining processing performed by the display controller 171 according to the third embodiment. The left figure in FIG. 10 is an example of a positional relation between the transverse section of the abdominal region of the subject P lying on the inspection bed and the orientation of the ultrasonic probe 11 pressed against the abdominal region. The right figure in FIG. 10 exemplifies a case in which the ultrasonic image obtained with the positional relation exemplified in the left figure is displayed in the display area of the monitor. In FIG. 10, the right direction is assumed to be the positive direction along the horizontal direction of the display area, and the downward direction is assumed to be the positive direction along the vertical direction of the display area.

As illustrated in the left figure in FIG. 10, exemplified is a case in which the ultrasonic image is generated in a state where the puncture needle 13 is attached to the ultrasonic probe 11 at the roll angle of “−60 degrees”. In this case, the display controller 171 receives, from the input device 16, an instruction to display the puncture guide line 28 at the roll angle of “−45 degrees” with respect to the vertical direction of the display area, and another instruction that the puncture needle 13 is attached at the roll angle of “−60 degrees” with respect to the ultrasonic probe 11.

As illustrated in the right figure in FIG. 10, the display controller 171 displays, on the monitor 17, the ultrasonic image on which the puncture guide line 28 is superimposed being tilted so that the puncture guide line 28 is at the roll angle of “−45 degrees” with respect to the vertical direction of the display area. Specifically, the puncture needle 13 is attached at the roll angle of “−60 degrees” with respect to the transmitting direction of the ultrasonic beam, and thus the display controller 171 displays the scanning range 24 of the ultrasonic image being tilted by the roll angle of “+15 degrees”.

As described above, the ultrasonic diagnostic device 1 according to the third embodiment receives, from the operator, an instruction on the display angle for displaying the puncture guide line 28 that indicates the puncture path of the puncture needle 13. The ultrasonic diagnostic device 1 then generates the ultrasonic image in which the puncture guide line 28 is superimposed on the scanning range 24 of the ultrasonic probe 11. The ultrasonic diagnostic device 1 then displays the scanning range of the ultrasonic image being tilted corresponding to the instructed display angle. Accordingly, the ultrasonic diagnostic device 1 according to the second embodiment can display the ultrasonic image being tilted so that the orientation of the puncture guide line is at the display angle intended by the operator.

In the third embodiment, described is a case in which both of the probe sensor 14a and the puncture needle sensor 14b are not included. However, both of them may be included. In this case, the ultrasonic diagnostic device 1 can display the ultrasonic image in a case in which free-puncturing is performed (the ultrasonic image exemplified in FIG. 9) being tilted so that the orientation of the puncture guide line is at the display angle intended by the operator.

That is, the ultrasonic diagnostic device 1 detects the position and the orientation of the ultrasonic probe 11 with respect to the gravity direction using the probe sensor 14a, and detects the position and the orientation of the puncture needle 13 with respect to the gravity direction using the puncture needle sensor 14b. The ultrasonic diagnostic device 1 then generates the ultrasonic image in which the puncture guide line 28 representing the puncture path of the puncture needle 13 is superimposed on the scanning range 24 of the ultrasonic probe 11 using the position and the orientation of the ultrasonic probe 11 and the position and the orientation of the puncture needle 13. The ultrasonic diagnostic device 1 displays the scanning range 24 of the ultrasonic image being tilted corresponding to the position and the orientation of the ultrasonic probe 11, the position and the orientation of the puncture needle 13, and the instructed display angle.

### Fourth Embodiment

In the above embodiment, described is a case of displaying the ultrasonic image while changing its orientation. However, the embodiment is not limited thereto. For example, in displaying the ultrasonic image while changing its orientation, the ultrasonic diagnostic device 1 may display another type of image while changing its orientation in parallel with the ultrasonic image. In the fourth embodiment, the following describes a case in which the ultrasonic diagnostic device 1 displays another type of image while changing its orientation in parallel with the ultrasonic image in displaying the ultrasonic image while changing its orientation.

The configuration of the ultrasonic diagnostic device 1 according to the fourth embodiment is basically the same as the configuration of the ultrasonic diagnostic device 1 described with reference to FIG. 1. However, part of the processing performed by the display controller 171 is different therebetween. Thus different points between the fourth embodiment and the first embodiment will be described, and the same points thereof will not be repeated.

The display controller 171 according to the fourth embodiment aligns the generated ultrasonic image with another type of image different from the generated ultrasonic image, and changes the orientation of the aligned another type of image to be displayed in displaying the generated ultrasonic image while changing its orientation.

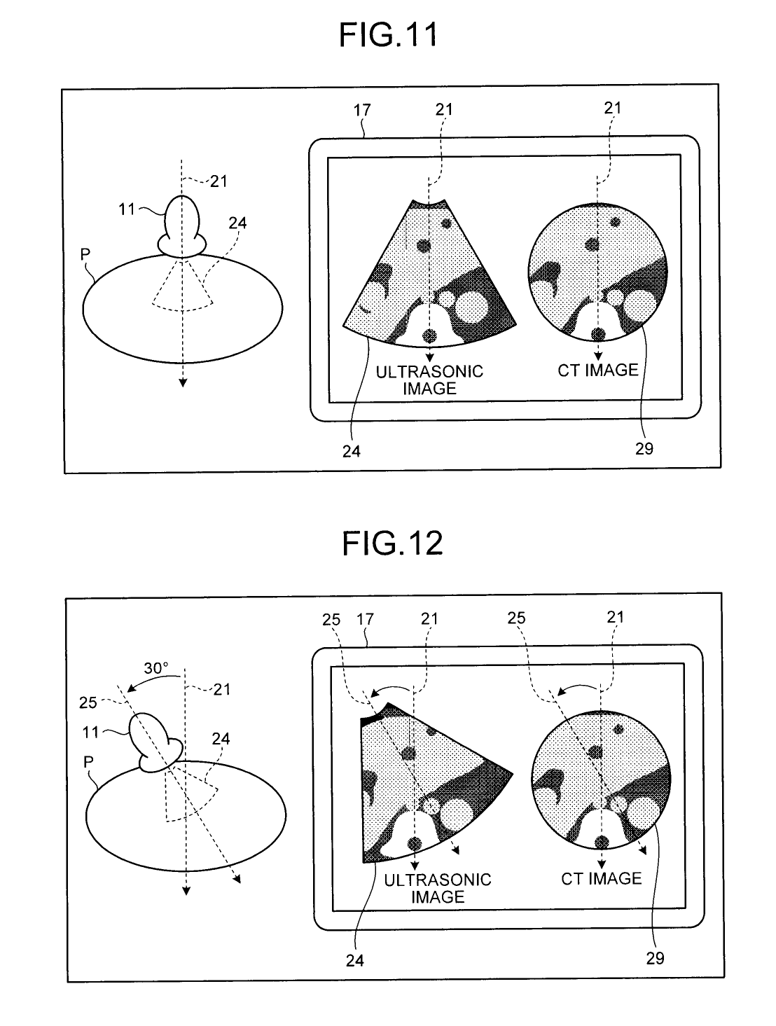

FIGS. 11 to 13 are diagrams for explaining processing performed by the display controller 171 according to the fourth embodiment. The left figure in FIGS. 11 to 13 is an example of a positional relation between the transverse section of the abdominal region of the subject P lying on the inspection bed and the orientation of the ultrasonic probe 11 pressed against the abdominal region. The right figure in FIGS. 11 to 13 exemplifies a case in which the ultrasonic image obtained with the positional relation exemplified in the left figure and a two-dimensional X-ray computed tomography (CT) image (hereinafter, abbreviated as a CT image) 29 corresponding to the ultrasonic image are displayed in the display area of the monitor 17. In FIGS. 11 to 13, the right direction is assumed to be the positive direction along the horizontal direction of the display area, and the downward direction is assumed to be the positive direction along the vertical direction of the display area.

With reference to FIG. 11, the following describes alignment processing performed by the display controller 171. As illustrated in FIG. 11, for example, the display controller 171 displays, on the monitor 17, the ultrasonic image generated in a state in which the ultrasonic probe 11 is pressed against the abdominal region of the subject P from directly above and the CT image 29 having substantially the same section. In this case, the CT image 29 displayed on the monitor 17 is a section generated through MPR processing from X-ray CT volume data obtained by photographing a target region of the subject P to be inspected. For example, the operator adjusts the position of the section for MPR processing via the input device 16 so that the CT image 29 representing the target region is displayed on the monitor 17. The display controller 171 then causes the image generation unit 140 to generate the CT image 29 obtained by cutting the X-ray CT volume data at the section adjusted by the operator (hereinafter, referred to as an initial section), and displays the CT image 29 on the monitor 17.

The operator readjusts the position of the initial section in the X-ray CT volume data so that the CT image 29 having substantially the same section as the ultrasonic image displayed on the monitor 17 is displayed. If the operator determines that the CT image 29 and the ultrasonic image displayed on the monitor 17 have substantially the same section, the operator pushes an enter button using the input device 16. When the enter button is pushed, the display controller 171 sets three-dimensional position information of the ultrasonic probe 11 acquired from the probe sensor 14a as initial position information. The display controller 171 determines the position of the initial section in the X-ray CT volume data at the time when the enter button is pushed as a final position of the initial section. Herein exemplified is a case in which the operator pushes the enter button to perform alignment. However, the embodiment is not limited thereto, and may be a case in which the CT image 29 having substantially the same section as the ultrasonic image is automatically extracted to perform alignment.

Thereafter the display controller 171 obtains movement information of a scanning section of the ultrasonic probe 11 from the initial position information and the position and the orientation of the ultrasonic probe 11 in the real space acquired from the probe sensor 14a, and changes the position of the initial section based on the acquired movement information to reset the section for MPR. The display controller 171 then causes the image generation unit 140 to generate the CT image 29 from the X-ray CT volume data using the reset section, and generates image data in which the CT image 29 and the ultrasonic image are arranged in parallel. In this way, the display controller 171 displays the ultrasonic image and the CT image 29 having substantially the same section as the ultrasonic image on a screen of the monitor 17 at the same time.

With reference to FIG. 12, the following describes processing of displaying the aligned another type of image while changing its orientation when the display controller 171 displays the ultrasonic image while changing its orientation. As illustrated in the left figure in FIG. 12, exemplified is a case in which the ultrasonic image is generated in a state where the ultrasonic probe 11 is pressed against the abdominal region of the subject P at the roll angle of “+30 degrees”. In this case, the display controller 171 extracts the roll angle of “+30 degrees” as the orientation of the ultrasonic probe 11.

As illustrated in the right figure in FIG. 12, when the display controller 171 displays, on the monitor 17, the scanning range 24 of the ultrasonic image being tilted by the roll angle of “+30 degrees”, the orientation of the aligned CT image 29 having substantially the same section is tilted in such a manner that they are linked to each other. As a result, each of the gravity direction 21 in the ultrasonic image and the gravity direction 21 in the CT image 29 is identical to the vertical direction of the display area. That is, the orientation of the tomogram of the subject P displayed in the ultrasonic image and the CT image 29 is identical to the orientation of the subject P in the real space, and thus the ultrasonic image and the CT image 29 in FIG. 12 are displayed so as to be intuitively recognizable to the viewer.

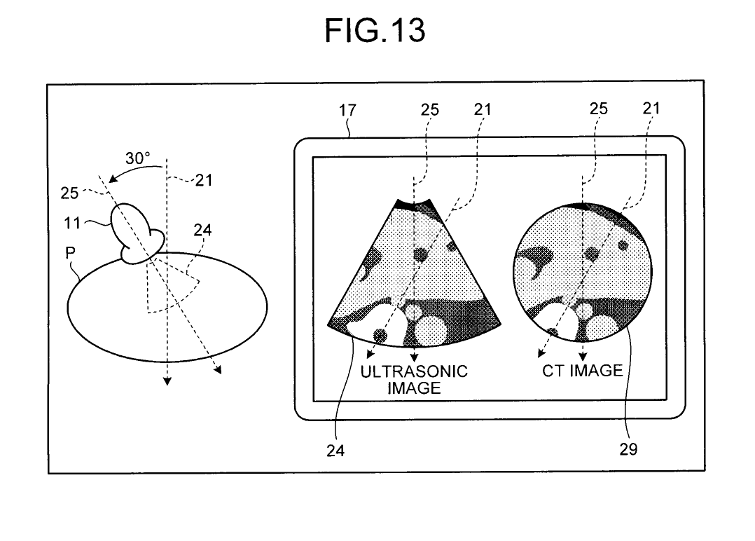

In contrast, FIG. 13 exemplifies a case in which the display controller 171 displays the ultrasonic image without changing its orientation. As illustrated in the left figure in FIG. 13, exemplified is a case in which the ultrasonic image is generated in a state where the ultrasonic probe 11 is pressed against the abdominal region of the subject P at the roll angle of “+30 degrees”. In this case, as illustrated in the right figure in FIG. 13, the orientation of the tomogram of the subject P displayed in the ultrasonic image and the CT image 29 is different from the orientation of the subject P in the real space, so that the ultrasonic image and the CT image 29 in FIG. 13 may give a sense of incongruity to the viewer.

As described above, the ultrasonic diagnostic device 1 according to the fourth embodiment aligns the ultrasonic image with another type of image different from the ultrasonic image, and displays the aligned another type of image while changing its orientation in displaying the ultrasonic image while changing its orientation. Due to this, the ultrasonic diagnostic device 1 can display the image aligned with the ultrasonic image so as to be intuitively recognizable.

In the fourth embodiment, described is a case of displaying the ultrasonic image and the CT image 29 while changing their orientations in such a manner that they are linked to each other. However, the embodiment is not limited thereto. For example, the ultrasonic diagnostic device 1 according to the fourth embodiment may align the ultrasonic image with another medical image such as a positron emission tomography (PET) image and a magnetic resonance (MR) image, and display both images while changing their orientations in such a manner that they are linked to each other. Alternatively, for example, the ultrasonic diagnostic device 1 may align the ultrasonic image with a certain image different from the ultrasonic image, and display both images while changing their orientations in such a manner that they are linked to each other. As a specific example, the ultrasonic diagnostic device 1 may align the ultrasonic image with an ultrasonic image of representative case or a past ultrasonic image of the same patient, and display both images while changing their orientations in such a manner that they are linked to each other. For example, the ultrasonic diagnostic device 1 may align the ultrasonic image with a picture depicting an anatomical positional relation between a lesion and another region, and display both images while changing their orientations in such a manner that they are linked to each other. The ultrasonic diagnostic device 1 may also display three or more images in such a manner that they are linked to each other.

### Fifth Embodiment

For example, the ultrasonic diagnostic device 1 may display the ultrasonic image being tilted so that the orientation of the puncture guide line is at a display angle intended by the operator in free-puncturing. In the fifth embodiment, described is a case in which the ultrasonic diagnostic device 1 displays the ultrasonic image being tilted so that the orientation of the puncture guide line is at the display angle intended by the operator in free-puncturing.

The configuration of the ultrasonic diagnostic device 1 according to the fifth embodiment is basically the same as the configuration of the ultrasonic diagnostic device 1 described with reference to FIG. 1. However, the configuration of the ultrasonic diagnostic device 1 according to the fifth embodiment is different from the configuration of the ultrasonic diagnostic device 1 described with reference to FIG. 1 in that the ultrasonic diagnostic device 1 according to the fifth embodiment includes both of the probe sensor 14a and the puncture needle sensor 14b, and part of the processing performed by the input device 16, the image generation unit 140, and the display controller 171 is different therebetween. Thus different points between the fifth embodiment and the first embodiment will be described, and the same points thereof will not be repeated.

The input device 16 according to the fifth embodiment receives, from the operator, an instruction on the display angle for displaying the puncture guide line 28 that indicates the puncture path of the puncture needle 13. For example, the input device 16 receives, from the operator, an instruction to display the puncture guide line 28 at the roll angle of “0 degrees” with respect to the vertical direction of the display area. The input device 16 then transmits the display angle instructed by the operator to the display controller 171.

The image generation unit 140 according to the fifth embodiment has the same function as that described in the second embodiment. That is, the image generation unit 140 according to the fifth embodiment generates the ultrasonic image in which the puncture guide line 28 representing the puncture path of the puncture needle 13 is superimposed on the scanning range of the ultrasonic probe 11 using the position and the orientation of the ultrasonic probe 11 and the position and the orientation of the puncture needle 13.

The display controller 171 according to the fifth embodiment displays the ultrasonic image being tilted corresponding to the instructed display angle.

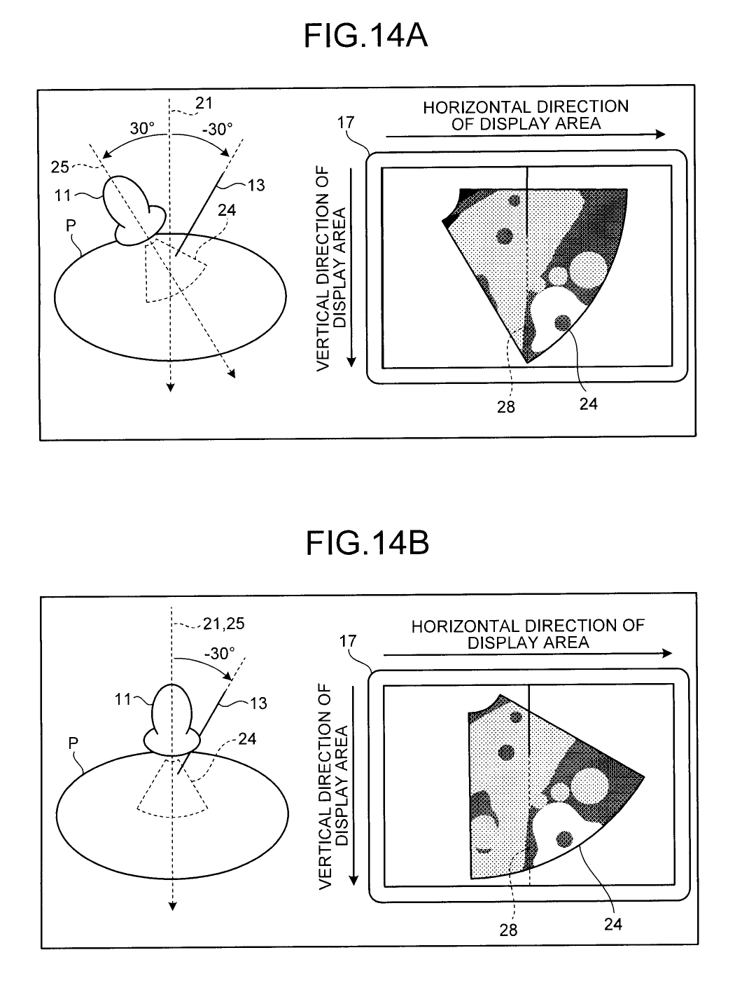

FIGS. 14A and 14B are diagrams for explaining processing performed by the image generation unit 140 and the display controller 171 according to the fifth embodiment. With reference to FIGS. 14A and 14B, described is a case in which the ultrasonic is displayed being tilted so that the orientation of the puncture guide line is at the roll angle of “0 degrees” with respect to the vertical direction of the display area in free-puncturing. Specifically, with reference to FIGS. 14A and 14B, described is a case in which the position and the orientation of the ultrasonic probe 11 are changed from the roll angle of “30 degrees” (FIG. 14A) to the roll angle of “0 degrees” (FIG. 14B) while free-puncturing is being performed. The left figure in FIGS. 14A and 14B is an example of a positional relation among the transverse section of the abdominal region of the subject P lying on the inspection bed, the position and the orientation of the ultrasonic probe 11 pressed against the abdominal region, and the position and the orientation of the puncture needle 13 inserted into the subject P. The right figure in FIGS. 14A and 14B exemplifies a case in which the ultrasonic image obtained with the positional relation exemplified in the left figure is displayed in the display area of the monitor. In FIGS. 14A and 14B, the right direction is assumed to be the positive direction along the horizontal direction of the display area, and the downward direction is assumed to be the positive direction along the vertical direction of the display area.

With reference to the left figure in FIG. 14A, the following describes processing performed by the image generation unit 140. For example, the image generation unit 140 obtains the position and the orientation of the ultrasonic probe 11 (at the roll angle of “30 degrees”) from the probe sensor 14a, and also obtains the position and the orientation of the puncture needle 13 (at the roll angle of “−30 degrees”) from the puncture needle sensor 14b. The image generation unit 140 then calculates the angle of the puncture guide line 28 with respect to the scanning range 24 based on the positional relation between the ultrasonic probe 11 and the puncture needle 13. In the example illustrated in the left figure in FIG. 14A, the ultrasonic probe 11 is pressed against the abdominal region of the subject P at the roll angle of “30 degrees” with respect to the gravity direction 21, and the puncture needle 13 is inserted into the abdominal region of the subject P at the roll angle of “−30 degrees” with respect to the gravity direction 21. In this case, the image generation unit 140 calculates the angle of the puncture guide line 28 with respect to the transmitting direction 25 of the ultrasonic beam for scanning the scanning range 24 to obtain the roll angle of “−60 degrees”. Subsequently, the image generation unit 140 calculates the intersection point of the scanning plane and the puncture guide line 28 in the scanning range 24. The intersection point is calculated because the puncture needle 13 does not necessarily present on the scanning plane and may intersect with the scanning plane in free-puncturing. The image generation unit 140 then generates the ultrasonic image in which the puncture guide line 28 is superimposed on the scanning range 24 so that the puncture guide line 28 in front of the scanning plane (intersection point) of the scanning range 24 is indicated by a solid line, and the puncture guide line 28 at the back thereof is indicated by a dashed line (the right figure in FIG. 14A). In this ultrasonic image, the angle of the puncture guide line 28 with respect to the transmitting direction 25 of the ultrasonic beam is “−60 degrees”.

Subsequently, with reference to the right figure in FIG. 14A, the following describes processing performed by the display controller 171. The display controller 171 receives, from the input device 16, an instruction to display the puncture guide line 28 at the roll angle of “0 degrees” (display angle) with respect to the vertical direction of the display area. The display controller 171 displays, on the monitor 17, the ultrasonic image generated by the image generation unit 140 while being tilted so that the puncture guide line 28 is at the roll angle of “0 degrees” with respect to the vertical direction of the display area.

When the position and the orientation of the ultrasonic probe 11 are changed from the roll angle of “30 degrees” (the left figure in FIG. 14A) to the roll angle of “0 degrees” (the left figure in FIG. 14B), the image generation unit 140 and the display controller 171 perform processing as follows.

With reference to the left figure in FIG. 14B, the following describes processing performed by the image generation unit 140. The image generation unit 140 obtains the position and the orientation of the ultrasonic probe 11 (the roll angle of “0 degrees”) from the probe sensor 14a, and obtains the position and the orientation of the puncture needle 13 (the roll angle of “−30 degrees”) from the puncture needle sensor 14b. In the example illustrated in the left figure in FIG. 14B, the ultrasonic probe 11 is pressed against the abdominal region of the subject P at the roll angle of “0 degrees” with respect to the gravity direction 21, and the puncture needle 13 is inserted into the abdominal region of the subject P at the roll angle of “−30 degrees” with respect to the gravity direction 21. In this case, the image generation unit 140 calculates the angle of the puncture guide line 28 with respect to the transmitting direction 25 of the ultrasonic beam for scanning the scanning range 24 to obtain the roll angle of “−30 degrees”, and generates the ultrasonic image in which the puncture guide line 28 is superimposed on the scanning range 24 similarly to the above processing. In this ultrasonic image, the angle of the puncture guide line 28 with respect to the transmitting direction 25 of the ultrasonic beam is “−30 degrees”.

Subsequently, with reference to the right figure in FIG. 14B, the following describes processing performed by the display controller 171. The display controller 171 displays, on the monitor 17, the ultrasonic image generated by the image generation unit 140 while being tilted so that the puncture guide line 28 is at the roll angle of “0 degrees” (display angle) with respect to the vertical direction of the display area.

In this way, the ultrasonic diagnostic device 1 according to the fifth embodiment can display the ultrasonic image being tilted so that the orientation of the puncture guide line is at the display angle intended by the operator in free-puncturing. For example, even when the position and the orientation of the ultrasonic probe 11 is changed from the roll angle of “30 degrees” (the left figure in FIG. 14A) to the roll angle of “0 degrees” (the left figure in FIG. 14B) in free-puncturing, the ultrasonic diagnostic device 1 can display the ultrasonic image with the orientation of the puncture guide line 28 at a certain angle constantly (the right figure in FIG. 14A and the right figure in FIG. 14B). Accordingly, the ultrasonic diagnostic device 1 can display the ultrasonic image so as to be intuitively recognizable using the puncture guide line as a reference even in free-puncturing.

### Sixth Embodiment

The first to fifth embodiments have been described above. In addition to the embodiments described above, various different embodiments may be employed.

Medical Image Processing Device

In the above embodiments, described is a case in which the ultrasonic diagnostic device 1 displays the generated ultrasonic image being tilted corresponding to the orientation of the ultrasonic probe 11 or the orientation of the puncture needle 13. However, the embodiment is not limited thereto. For example, a medical image processing device can display the ultrasonic image being tilted corresponding to the orientation of the ultrasonic probe 11 or the orientation of the puncture needle 13 in reproducing the ultrasonic image generated by the ultrasonic diagnostic device 1.

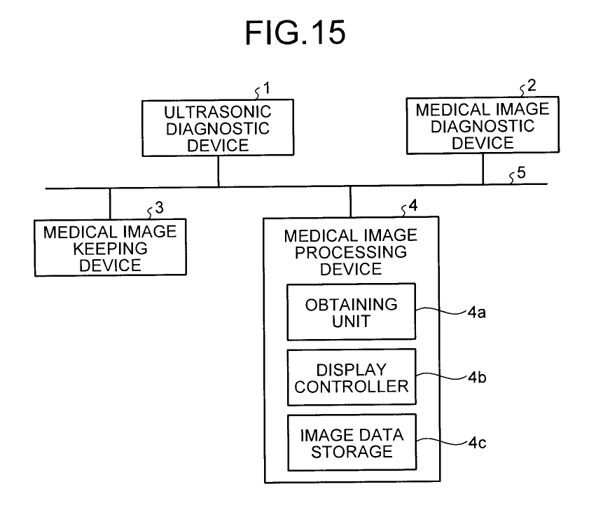

FIG. 15 is a diagram for explaining a configuration of a medical information system according to a sixth embodiment. As illustrated in FIG. 15, the medical information system according to the sixth embodiment includes the ultrasonic diagnostic device 1, a medical image diagnostic device 2, a medical image keeping device 3, and a medical image processing device 4. The devices can directly or indirectly communicate with each other, for example, via an in-hospital local area network (LAN) 5 installed in a hospital. For example, when a picture archiving and communication system (PACS) is introduced in the medical information system, the devices transmit or receive medical image data and the like to/from each other in compliance with the digital imaging and communications in medicine (DICOM) standard.

The medical image diagnostic device 2 is a device such as an X-ray diagnostic device, an X-ray CT device, an MRI device, an ultrasonic diagnostic device, a single photon emission computed tomography (SPECT) device, a positron emission computed tomography (PET) device, a SPECT-CT device in which the SPECT device and the X-ray CT device are integrated, a PET-CT device in which the PET device and the X-ray CT device are integrated, and a specimen inspecting device. For example, the medical image diagnostic device 2 photographs a subject corresponding to an operation by a radiographer who photographs the subject, and generates medical image data and an inspection result.

The medical image keeping device 3 is a device that keeps medical image data. For example, the medical image keeping device 3 includes a database for storing the medical image data, and stores and keeps the medical image data and the inspection result generated by the medical image diagnostic device 2 in the database.

The medical image processing device 4 is an image processing device that performs image processing on the medical image data. For example, the medical image processing device 4 obtains the medical image data and the inspection result from the medical image keeping device 3, and displays the acquired medical image data and the inspection result on the monitor.

As illustrated in FIG. 15, the medical image processing device 4 includes an obtaining unit 4a, a display controller 4b, and image data storage 4c.

The obtaining unit 4a obtains the ultrasonic image data generated by the ultrasonic diagnostic device 1 to store the obtained data in the image data storage 4c. For example, the obtaining unit 4a obtains the ultrasonic image and the orientation of the ultrasonic probe 11 or the orientation of the puncture needle 13 at the time when the ultrasonic image is generated. The obtaining unit 4a then stores the obtained ultrasonic image and the orientation of the ultrasonic probe 11 or the orientation of the puncture needle 13 at the time when the ultrasonic image is generated in a manner associated with each other for each frame in the image data storage 4c.

The display controller 4b displays the ultrasonic image generated by the ultrasonic diagnostic device 1 being tilted corresponding to the orientation of the ultrasonic probe 11 or the orientation of the puncture needle 13 at the timing when the ultrasonic image is generated. The display controller 4b has the same function as the display controller 171 described with reference to FIG. 1.

The image data storage 4c stores therein the ultrasonic image and the orientation of the ultrasonic probe 11 or the orientation of the puncture needle 13 at the time when the ultrasonic image is generated in a manner associated with each other for each frame.

Accordingly, the medical image processing device 4 according to the sixth embodiment can display the ultrasonic image generated by the ultrasonic diagnostic device 1 so as to be intuitively recognizable.

The description with reference to FIG. 15 is merely an example. For example, when free-puncturing is performed as described in the second embodiment, the position and the orientation of the ultrasonic probe 11 and the position and the orientation of the puncture needle 13 may be stored for each frame. Specifically, the obtaining unit 4a obtains the ultrasonic image and the position and the orientation of the ultrasonic probe 11 and the position and the orientation of the puncture needle 13 at the time when the ultrasonic image is generated. The obtaining unit 4a then stores the obtained ultrasonic image and the position and the orientation of the ultrasonic probe 11 and the position and the orientation of the puncture needle 13 at the time when the ultrasonic image is generated in a manner associated with each other for each frame in the image data storage 4c. Due to this, the ultrasonic diagnostic device 1 can display the ultrasonic image so as to be intuitively recognizable and display the position and the orientation of the puncture needle 13 at the point even when free-puncturing is performed.

For example, the image data storage 4c does not necessarily store the orientation of the ultrasonic probe 11 or the orientation of the puncture needle 13 at the time when the ultrasonic image is generated for each frame. That is, the image data storage 4c may store the orientation of the ultrasonic probe 11 or the orientation of the puncture needle 13 in a certain period in association with the ultrasonic image data of multiple frames included in the certain period. In this case, the display controller 4b displays the ultrasonic image of multiple frames included in the certain period while changing its orientation, corresponding to the orientation of the ultrasonic probe 11 or the orientation of the puncture needle 13 stored in the image data storage 4c.

Specifically, for example, the orientation of the ultrasonic probe 11 or the orientation of the puncture needle 13 is kept at a certain angle constantly in many cases while puncturing is being performed. That is, while puncturing is being performed, the orientation of the ultrasonic probe 11 and the orientation of the puncture needle 13 associated with the ultrasonic image data of multiple frames arranged in a time series manner are at substantially the same angle. In such a case, a unique orientation of the ultrasonic probe 11 or a unique orientation of the puncture needle 13 may be stored for the ultrasonic image data of multiple frames. For example, in the medical image processing device 4, the obtaining unit 4a calculates an average angle (average value) of the orientation of the ultrasonic probe 11 associated with the ultrasonic image data of multiple frames. The obtaining unit 4a then stores the ultrasonic image data of multiple frames and the calculated average angle in a manner associated with each other in the image data storage 4c. Accordingly, for example, the medical image processing device 4 can reduce a data amount to be stored, or reduce a processing load of display. The orientation of the ultrasonic probe 11 and the orientation of the puncture needle 13 associated with the ultrasonic image data of multiple frames are at the same angle not only in puncturing but also in a case in which a physician performs another treatment or performs observation for a certain period of time.

That is, in the medical image diagnostic device 2, the image data storage 4c stores therein the ultrasonic image and the orientation of the ultrasonic probe 11 or the orientation of the puncture needle inserted into the subject in a manner associated with each other. The display controller 4b displays the ultrasonic image while changing its orientation, corresponding to the orientation of the ultrasonic probe 11 or the orientation of the puncture needle 13 stored in the image data storage 4c.

Parallel Display

For example, the ultrasonic diagnostic device 1 may display an image obtained by changing the orientation of the ultrasonic image corresponding to the orientation of the ultrasonic probe 11 or the orientation of the puncture needle 13, and an image before the change in parallel at the same time. As a specific example, the ultrasonic diagnostic device 1 may display the right figure in FIG. 5 and the right figure in FIG. 3B in parallel at the same time.

Ultrasonic Probe Including Puncture Guide Rail

In the above embodiments, described is a case in which the puncture needle 13 is attached to the ultrasonic probe 11 at a certain position and orientation using the puncture adapter 12. However, the embodiment is not limited thereto. For example, the ultrasonic probe 11 may include the puncture guide rail. The puncture guide rail is provided to the ultrasonic probe 11 at a certain position and orientation. Accordingly, in the ultrasonic diagnostic device 1, the puncture needle 13 can be attached to the ultrasonic probe 11 at a certain position and orientation by using the ultrasonic probe 11 including the puncture guide rail without using the puncture adapter 12. The puncture guide rail is also referred to as an inserting part.

Detection of Puncture Needle Through Image Processing

In the above embodiments, described is a case in which the orientation of the puncture needle 13 is detected by the sensor provided to the ultrasonic probe 11 or the puncture needle 13. Alternatively, the orientation of the puncture needle depicted on the ultrasonic image may be detected through image processing, and the orientation of the ultrasonic image may be changed and displayed corresponding to the detected orientation. Specifically, for example, the ultrasonic diagnostic device 1 or the medical image processing device 4 includes an image processing unit (not illustrated). The image processing unit recognizes a part of the ultrasonic image having a luminance value equal to or larger than a certain threshold as an image related to the puncture needle 13, and detects the orientation of the puncture needle 13 in the ultrasonic image. The display controller 171 displays, on a certain display module, the ultrasonic image while changing its orientation so that the orientation of the puncture needle 13 detected by the image processing unit is substantially identical to a display angle set by the operator in advance. In this case, the sensor is not required to be provided to the ultrasonic probe 11 or the puncture needle 13, and the configuration of the ultrasonic probe 11 or the puncture needle 13 can be further simplified.

According to at least one of the embodiments described above, the ultrasonic image can be displayed so as to be intuitively recognizable.

The components of the ultrasonic diagnostic device 1 exemplified in FIG. 1 are merely conceptual, and the ultrasonic diagnostic device 1 does not necessarily physically include the components as illustrated in FIG. 1. That is, specific forms of distribution and integration of the components of the ultrasonic diagnostic device 1 are not limited to those illustrated in FIG. 1. By way of example, in the display controller 171 according to the fourth embodiment, a processing unit different from the display controller 171 may perform alignment processing.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An ultrasonic diagnostic device comprising:
generation circuitry configured to generate an ultrasonic image based on reflected waves received by an ultrasonic probe;
detection circuitry configured to detect an orientation of a puncture needle inserted into a subject; and
display control circuitry configured to display the generated ultrasonic image while changing an orientation of the image, corresponding to the detected orientation of the puncture needle.

2. The ultrasonic diagnostic device according to claim 1, wherein
the detection circuitry is configured to detect, as the orientation of the puncture needle, the orientation of the puncture needle inserted from a puncture adapter mounted on the ultrasonic probe or an inserting part of the ultrasonic probe in a real space, and
the display control circuitry is configured to display the ultrasonic image while changing the orientation of the image, using the orientation of the puncture needle and the orientation of the puncture adapter or the inserting part with respect to the ultrasonic probe.

3. The ultrasonic diagnostic device according to claim 1, wherein
the detection circuitry is configured to detect a position and an orientation of the ultrasonic probe with respect to a gravity direction using a sensor provided to the ultrasonic probe, and detect a position and an orientation of the puncture needle with respect to the gravity direction using a sensor provided to the puncture needle, and
the generation circuitry is configured to generate an ultrasonic image in which a puncture guide line representing a puncture path of the puncture needle is superimposed on a scanning range of the ultrasonic probe, using the position and the orientation of the ultrasonic probe and the position and the orientation of the puncture needle.

4. The ultrasonic diagnostic device according to claim 1, wherein the generation circuitry is configured to generate, as the ultrasonic image, an ultrasonic image in which a puncture guide line representing a puncture path of the puncture needle is superimposed on a scanning range of the ultrasonic probe.

5. The ultrasonic diagnostic device according to claim 3, further comprising:
reception circuitry configured to receive an instruction on a display angle for displaying the puncture guide line from an operator, wherein
the display control circuitry is configured to display the ultrasonic image on which the puncture guide line is superimposed while changing the orientation of the image so that the puncture guide line is displayed at the display angle.

6. The ultrasonic diagnostic device according to claim 4, further comprising:
reception circuitry configured to receive an instruction on a display angle for displaying the puncture guide line from an operator, wherein
the display control circuitry is configured to display the ultrasonic image on which the puncture guide line is superimposed while changing the orientation of the image so that the puncture guide line is displayed at the display angle.

7. An ultrasonic diagnostic device comprising:
generation circuitry configured to generate an ultrasonic image based on reflected waves received by an ultrasonic probe;
detection circuitry configured to detect an orientation of the ultrasonic probe or an orientation of a puncture needle inserted into a subject; and
display control circuitry configured to align the ultrasonic image with a certain image different from the ultrasonic image, and display the ultrasonic image and the certain image while changing orientations of the images, corresponding to the orientation of the ultrasonic probe or the orientation of the puncture needle detected by the detection circuitry.

8. A medical image processing device comprising:
storage circuitry configured to store therein an ultrasonic image and an orientation of an ultrasonic probe or an orientation of a puncture needle inserted into a subject in a manner associated with each other; and
display control circuitry configured to display the ultrasonic image while changing an orientation of the image, corresponding to the orientation of the ultrasonic probe or the orientation of the puncture needle stored in the storage circuitry.

9. The medical image processing device according to claim 8, wherein
the storage circuitry is configured to store the ultrasonic image and the orientation of the ultrasonic probe or the orientation of the puncture needle at the time when the ultrasonic image is generated in a manner associated with each other for each frame, and
the display control circuitry is configured to display the ultrasonic image while changing the orientation of the image, corresponding to the orientation of the ultrasonic probe or the orientation of the puncture needle associated with the ultrasonic image for each frame.

10. The medical image processing device according to claim 8, wherein
the storage circuitry is configured to store therein an ultrasonic image of multiple frames included in a certain period and the orientation of the ultrasonic probe or the orientation of the puncture needle in the certain period in a manner associated with each other, and
the display control circuitry is configured to display the ultrasonic image of multiple frames included in the certain period while changing orientations of the image, corresponding to the orientation of the ultrasonic probe or the orientation of the puncture needle stored in the storage circuitry.
